(19) United States
(12) Patent Application Publication
(10) Pub. No.: US 2020/0367858 A1
(43) Pub. Date: Nov. 26, 2020

(54) DUAL LAYER ULTRASONIC TRANSDUCER

(71) Applicant: InvenSense, Inc., San Jose, CA (US)

(72) Inventors: Leonardo BALDASARRE, Gavirate (IT); Mei-Lin CHAN, Milpitas, CA (US)

(73) Assignee: InvenSense, Inc., San Jose, CA (US)

(21) Appl. No.: 16/879,519

(22) Filed: May 20, 2020

Related U.S. Application Data
(60) Provisional application No. 62/850,448, filed on May 20, 2019.

(51) Int. Cl. A61B 8/00; B06B 1/06

(52) U.S. Cl. CPC A61B 8/4494; A61B 8/08; B06B 1/0607

(57) ABSTRACT

An ultrasonic transducer device including a substrate, an edge support structure connected to the substrate, and a membrane connected to the edge support structure such that a cavity is defined between the membrane and the substrate, the membrane configured to allow movement at ultrasonic frequencies. The membrane includes a first piezoelectric layer having a first surface and a second surface, a second piezoelectric layer having a first surface and a second surface, wherein the second surface of the first piezoelectric layer faces the first surface of the second piezoelectric layer, a first electrode coupled to the first surface of the first piezoelectric layer, a second electrode coupled to the second surface of the second piezoelectric layer, and a third electrode between the first piezoelectric layer and the second piezoelectric layer.

### RELATED APPLICATIONS

This application claims priority to and the benefit of co-pending U.S. Patent Provisional Patent Application 62/850,448, filed on May 20, 2019, entitled “NEXT GENERATION FINGERPRINT SENSOR,” by Apte et al., having Attorney Docket No. IVS-836-PR, and assigned to the assignee of the present application, which is incorporated herein by reference in its entirety.

### BACKGROUND

Piezoelectric materials facilitate conversion between mechanical energy and electrical energy. Moreover, a piezoelectric material can generate an electrical signal when subjected to mechanical stress, and can vibrate when subjected to an electrical voltage. Piezoelectric materials are widely utilized in piezoelectric ultrasonic transducers to generate acoustic waves based on an actuation voltage applied to electrodes of the piezoelectric ultrasonic transducer.

### BRIEF DESCRIPTION OF THE DRAWINGS

The accompanying drawings, which are incorporated in and form a part of the Description of Embodiments, illustrate various embodiments of the subject matter and, together with the Description of Embodiments, serve to explain principles of the subject matter discussed below. Unless specifically noted, the drawings referred to in this Brief Description of Drawings should be understood as not being drawn to scale. Herein, like items are labeled with like item numbers.

FIG. 1 is a diagram illustrating a dual layer ultrasonic transducer device, according to some embodiments.

FIG. 2A is a diagram illustrating an example transmit operation of a dual layer ultrasonic transducer device, where the two piezoelectric layers are activated during the transmit operation, according to some embodiments.

FIG. 2B is a diagram illustrating an example receive operation of a dual layer ultrasonic transducer device, where the two piezoelectric layers are activated during the receive operation, according to some embodiments.

FIG. 3A is a diagram illustrating an example transmit operation of a dual layer ultrasonic transducer device, where one piezoelectric layer is activated during the transmit operation, according to some embodiments.

FIG. 3B is a diagram illustrating an example receive operation of a dual layer ultrasonic transducer device, where one piezoelectric layer is activated during the receive operation, according to some embodiments.

FIG. 4 is a diagram illustrating a dual layer ultrasonic transducer device including a mode control switch for switching between activating one piezoelectric layer and activating two piezoelectric layers during a transmit and/or receive operation, according to various embodiments.

FIG. 5 is a diagram illustrating a dual layer ultrasonic transducer device having an interior support structure coupled to the substrate and the membrane, according to some embodiments.

FIG. 6 is a diagram illustrating a dual layer ultrasonic transducer device having piezoelectric layers comprised of different materials, according to some embodiments.

FIG. 7A is a diagram illustrating an example transmit operation of a dual layer ultrasonic transducer device having piezoelectric layers comprised of different materials, according to some embodiments.

FIG. 7B is a diagram illustrating an example receive operation of a dual layer ultrasonic transducer device having piezoelectric layers comprised of different materials, according to some embodiments.

FIG. 8 is a diagram illustrating a dual layer ultrasonic transducer device having piezoelectric layers having different thicknesses, according to some embodiments.

FIG. 9 is a diagram illustrating a dual layer ultrasonic transducer device having a buffer layer between the two piezoelectric layers, according to some embodiments.

FIG. 10A is a diagram illustrating an example transmit operation of a dual layer ultrasonic transducer device having a buffer layer between the two piezoelectric layers, according to some embodiments.

FIG. 10B is a diagram illustrating an example receive operation of a dual layer ultrasonic transducer device having a buffer layer between the two piezoelectric layers, according to some embodiments.

FIG. 11 is a diagram illustrating a dual layer ultrasonic transducer device having a buffer layer between the two piezoelectric layers and an interior support structure coupled to the substrate and the membrane, according to some embodiments.

### DESCRIPTION OF EMBODIMENTS

The following Description of Embodiments is merely provided by way of example and not of limitation. Furthermore, there is no intention to be bound by any expressed or implied theory presented in the preceding background or in the following Description of Embodiments.

Reference will now be made in detail to various embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications and equivalents, which may be included within the spirit and scope the various embodiments as defined by the appended claims. Furthermore, in this Description of Embodiments, numerous specific details are set forth in order to provide a thorough understanding of embodiments of the present subject matter. However, embodiments may be practiced without these specific details. In other instances, well known methods, procedures, components, and circuits have not been described in detail as not to unnecessarily obscure aspects of the described embodiments.

### Notation and Nomenclature

Some portions of the detailed descriptions which follow are presented in terms of procedures, logic blocks, processing and other symbolic representations of operations on data within an electrical device. These descriptions and representations are the means used by those skilled in the data processing arts to most effectively convey the substance of their work to others skilled in the art. In the present application, a procedure, logic block, process, or the like, is conceived to be one or more self-consistent procedures or instructions leading to a desired result. The procedures are those requiring physical manipulations of physical quantities. Usually, although not necessarily, these quantities take the form of acoustic (e.g., ultrasonic) signals capable of being transmitted and received by an electronic device and/or electrical or magnetic signals capable of being stored, transferred, combined, compared, and otherwise manipulated in an electrical device.

It should be borne in mind, however, that all of these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “transmitting,” “receiving,” “sensing,” “generating,” “imaging,” or the like, refer to the actions and processes of an electronic device such as an ultrasonic transducer or an array of ultrasonic transducers.

Embodiments described herein may be discussed in the general context of processor-executable instructions residing on some form of non-transitory processor-readable medium, such as program modules, executed by one or more computers or other devices for controlling operation of one or more dual layer ultrasonic transducer devices. Various techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, perform one or more of the methods described herein. The non-transitory processor-readable data storage medium may form part of a computer program product, which may include packaging materials.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor.

Various embodiments described herein may be executed by one or more processors, such as one or more, sensor processing units (SPUs), host processor(s) or core(s) thereof, digital signal processors (DSPs), general purpose microprocessors, application specific integrated circuits (ASICs), application specific instruction set processors (ASIPs), field programmable gate arrays (FPGAs), a programmable logic controller (PLC), a complex programmable logic device (CPLD), a discrete gate or transistor logic, discrete hardware components, or any combination thereof designed to perform the functions described herein, or other equivalent integrated or discrete logic circuitry. The term “processor,” as used herein may refer to any of the foregoing structures or any other structure suitable for implementation of the techniques described herein. As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Moreover, processors can exploit nano-scale architectures such as, but not limited to, molecular and quantum-dot based transistors, switches and gates, in order to optimize space usage or enhance performance of user equipment. A processor may also be implemented as a combination of computing processing units.

In addition, in some aspects, the functionality described herein may be provided within dedicated software modules or hardware modules configured as described herein. Also, the techniques could be fully implemented in one or more circuits or logic elements. A general purpose processor may be a microprocessor, but in the alternative, the processor may be any conventional processor, controller, microcontroller, or state machine. A processor may also be implemented as a combination of computing devices, e.g., a combination of an SPU and a microprocessor, a plurality of microprocessors, one or more microprocessors in conjunction with an SPU core, or any other such configuration.

### Overview of Discussion

Discussion includes a description of an example dual layer ultrasonic transducer, in accordance with various embodiments. Various different embodiments of dual layer ultrasonic transducers are then described, including dual layer ultrasonic transducers having an interior support structure, dual layer ultrasonic transducers including a buffer layer between the dual piezoelectric layers, and other embodiments of dual layer ultrasonic transducers.

A conventional piezoelectric ultrasonic transducer able to generate and detect pressure waves can include a membrane including piezoelectric material and electrodes combined with a cavity beneath the electrodes. The membrane of ultrasonic transducers may include other layers, such as a mechanical support layer, acoustic coupling layers, or other layers. Miniaturized versions are referred to as piezoelectric micromachined ultrasonic transducers (PMUT). An example of a single layer ultrasonic transducer device is described in U.S. Pat. No. 10,656,255.

Embodiments described herein relate to a dual layer ultrasonic transducer for ultrasonic wave generation and sensing. In accordance with various embodiments, the ultrasonic transducer device includes a substrate, an edge support structure connected to the substrate, and a membrane connected to the edge support structure such that a cavity is defined between the membrane and the substrate, where the membrane configured to allow movement at ultrasonic frequencies. The membrane includes two piezoelectric layers and at least three electrodes, wherein each piezoelectric layer is between two electrodes, and one electrode is between the two piezoelectric layers. In some embodiments, the piezoelectric layers have different thicknesses. In some embodiments, the piezoelectric layers are comprised of different materials.

In some embodiments, the ultrasonic transducer device includes an interior support structure disposed within the cavity and connected to the substrate and the membrane. In some embodiments, the membrane further includes a buffer layer between the first piezoelectric layer and the second piezoelectric layer and fourth electrode, wherein each piezoelectric layer and the buffer layer is between two electrodes.

The described dual layer ultrasonic transducer device can be used for generation of acoustic signals or measurement of acoustically sensed data in various applications, such as, but not limited to, medical applications, security systems, biometric systems (e.g., fingerprint sensors and/or motion/gesture recognition sensors), mobile communication systems, industrial automation systems, consumer electronic devices, robotics, etc., for example, using multiple dual layer ultrasonic transducer devices operating collectively. In one embodiment, the dual layer ultrasonic transducer device can facilitate ultrasonic signal generation and sensing. Moreover, embodiments describe herein provide a sensing component including a substrate including a two-dimensional (or one-dimensional) array of dual layer ultrasonic transducer devices.

### Dual Layer Ultrasonic Transducer

Embodiments described herein provide dual layer ultrasonic transducers. One or more embodiments are now described with reference to the drawings, wherein like reference numerals are used to refer to like elements throughout. In the following description, for purposes of explanation, numerous specific details are set forth in order to provide a thorough understanding of the various embodiments. It may be evident, however, that the various embodiments can be practiced without these specific details. In other instances, well-known structures and devices are shown in block diagram form in order to facilitate describing the embodiments in additional detail.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form. In addition, the word “coupled” is used herein to mean direct or indirect electrical or mechanical coupling. In addition, the word “example” is used herein to mean serving as an example, instance, or illustration.

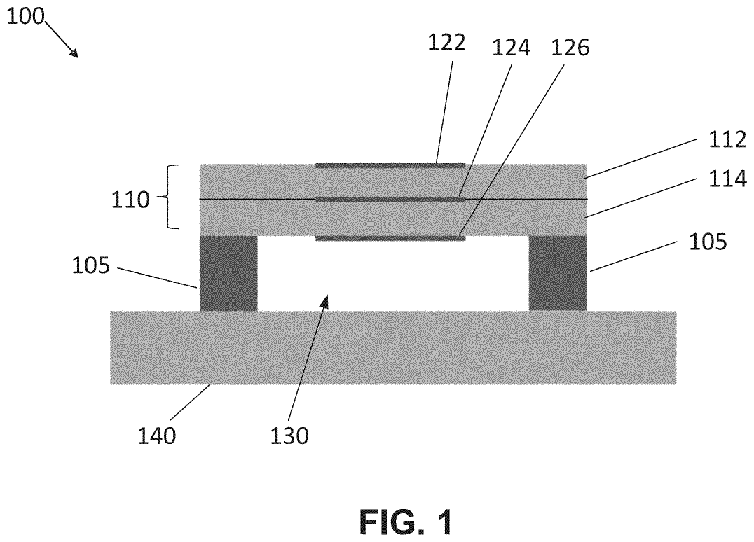

FIG. 1 is a diagram illustrating a dual layer ultrasonic transducer device 100, according to some embodiments. Dual layer ultrasonic transducer device 100 includes a membrane 110 positioned over a substrate 140 to define a cavity 130. In one embodiment, membrane 110 is attached to a surrounding edge support 105. In one embodiment, edge support 105 is connected to an electric potential for connecting to electrodes 122, 124, and or 126. Edge support 105 may be made of electrically conducting materials, such as and without limitation, aluminum, molybdenum, or titanium. Edge support 105 may also be made of dielectric materials, such as silicon dioxide, silicon nitride or aluminum oxide that have electrical connections along the sides or in vias through edge support 105, for electrically coupling electrode 122, 124, and/or 126 to electrical wiring in substrate 140. For example, substrate 140 may include terminals for electrically coupling electrodes 122, 124, and/or 126 to control circuitry.

In various embodiments, substrate 140 may include at least one of, and without limitation, silicon or silicon nitride. It should be appreciated that substrate 140 may include electrical wirings and connection, such as aluminum or copper. In one embodiment, substrate 140 includes a CMOS logic wafer bonded to edge support 105. In one embodiment, the membrane 110 comprises multiple piezoelectric layers. In an example embodiment, the membrane 110 includes piezoelectric layers 112 and 114, and electrodes 122, 124, and 126, with electrodes 122 and 124 on opposing sides of piezoelectric layer 112 and electrodes 124 and 126 on opposing sides of piezoelectric layer 114, where electrode 124 is between piezoelectric layers 112 and 114. While embodiments described herein are directed toward a dual layer ultrasonic transducer device including two piezoelectric layers, it should be appreciated that the principles described herein allow for the use of more than two piezoelectric layers, and that in some conceivable embodiments a multi-layer ultrasonic transducer device including more than two piezoelectric layers may be utilized. It should be appreciated that, in various embodiments, dual ultrasonic transducer device 100 is a microelectromechanical (MEMS) device. In accordance with various embodiments, piezoelectric layers 112 and 114 have thicknesses in the range of one to ten microns (e.g., two microns, such that membrane 110 has a thickness of four microns).

It should be appreciated that, dual layer ultrasonic transducer device 100 (and membrane 110) can be one of many types of geometric shapes (e.g., ring, circle, square, octagon, hexagon, etc.). For example, a sensing device may include an array of dual layer ultrasonic transducer devices 100. In some embodiments, the dual layer ultrasonic transducer devices 100 can be of a shape that allows for close adjacent placement of dual layer ultrasonic transducer devices 100. In some embodiments, adjacent dual layer ultrasonic transducer devices 100 within an array may share edge support structures 105. In other embodiments, adjacent dual layer ultrasonic transducer devices 100 within an array are electrically and physically isolated from each other (e.g., separated by a gap).

It should be appreciated that in accordance with various embodiments, membrane 110 can also include other layers (not shown), such a mechanical support layer, e.g., stiffening layer, and an acoustic coupling layer. The mechanical support layer is configured to mechanically stiffen the layers of membrane 110. The mechanical support layer can be above or below membrane 110. In various embodiments, the mechanical support layer may include at least one of, and without limitation, silicon, silicon oxide, silicon nitride, aluminum, molybdenum, titanium, etc. The acoustic coupling layer is for supporting transmission of acoustic signals, and, if present, is above membrane 110. It should be appreciated that acoustic coupling layer can include air, liquid, gel-like materials, or other materials for supporting transmission of acoustic signals.

In some embodiments, a plurality of dual layer ultrasonic transducer devices 100 are comprised within a two-dimensional (or one-dimensional) array of dual layer ultrasonic transducer devices. In such embodiments, the array of dual layer ultrasonic transducer devices 100 may be coupled to a platen layer above an acoustic coupling layer for containing the acoustic coupling layer and providing a contact surface for a finger or other sensed object with the array of dual layer ultrasonic transducer devices 100. It should be appreciated that, in various embodiments, the acoustic coupling layer provides a contact surface, such that a platen layer is optional. It should be appreciated that the contact surface can be flat or of a varying thickness (e.g., curved).

The described dual layer ultrasonic transducer device 100 is capable of generating and receiving ultrasonic signals. An object in a path of the generated ultrasonic signals can create a disturbance (e.g., changes in frequency or phase, reflection signal, echoes, etc.) that can then be sensed. The interference can be analyzed to determine physical parameters such as (but not limited to) distance, density and/or speed of the object. As an example, the dual layer ultrasonic transducer device 100 can be utilized in various applications, such as, but not limited to, fingerprint or physiologic sensors suitable for wireless devices, industrial systems, automotive systems, robotics, telecommunications, security, medical devices, etc. For example, the dual layer ultrasonic transducer device 100 can be part of a sensor array comprising a plurality of ultrasonic transducers deposited on a wafer, along with various logic, control and communication electronics. A sensor array may comprise homogenous or identical dual layer ultrasonic transducer devices 100, or a number of different or heterogonous device structures.

In various embodiments, the dual layer ultrasonic transducer device 100 employs piezoelectric layers 112 and 114, comprised of materials such as, but not limited to, aluminum nitride (AlN), scandium doped aluminum nitride (ScAlN), lead zirconate titanate (PZT), quartz, polyvinylidene fluoride (PVDF), and/or zinc oxide, to facilitate both acoustic signal production (transmitting) and sensing (receiving). The piezoelectric layers 112 and/or 114 can generate electric charges under mechanical stress and conversely experience a mechanical strain in the presence of an electric field. For example, piezoelectric layers 112 and/or 114 can sense mechanical vibrations caused by an ultrasonic signal and produce an electrical charge at the frequency (e.g., ultrasonic frequency) of the vibrations. Additionally, piezoelectric layers 112 and/or 114 can generate an ultrasonic wave by vibrating in an oscillatory fashion that might be at the same frequency (e.g., ultrasonic frequency) as an input current generated by an alternating current (AC) voltage applied across the piezoelectric layers 112 and/or 114. It should be appreciated that piezoelectric layers 112 and 114 can include almost any material (or combination of materials) that exhibits piezoelectric properties. The polarization is directly proportional to the applied stress and is direction dependent so that compressive and tensile stresses results in electric fields of opposite polarizations.

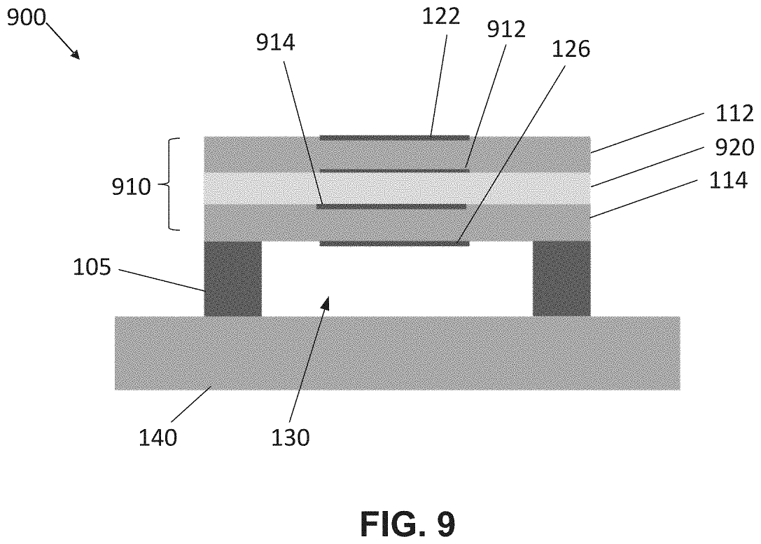

Further, dual layer ultrasonic transducer device 100 comprises electrodes 122, 124, and 126 that supply and/or collect the electrical charge to/from piezoelectric layers 112 and 114. Electrodes 122, 124, and 126 can be connected to substrate 140 or the underlying circuitry via one or more terminals on substrate 140. Depending on the mode of operation, two or more electrodes may share a single terminal. It should be appreciated that electrodes 122, 124, and 126 can be continuous and/or patterned electrodes (e.g., in a continuous layer and/or a patterned layer). As an example, electrodes 122, 124, and 126 can be comprised of almost any metal layers, such as, but not limited to, aluminum (Al), titanium (Ti), Molybdenum (Mo), etc. In some embodiments, dual layer ultrasonic transducer device 100 also includes a fourth electrode, as illustrated in FIG. 9 and described below.

In accordance with various embodiments, electrodes 122, 124, and/or 126 can be patterned in particular shapes (e.g., ring, circle, square, octagon, hexagon, etc.) that are defined in-plane with the membrane 110. Electrodes 122, 124, and 126 can be placed at a maximum strain area of the membrane 110 or placed close to edge support 105. Furthermore, in one example, electrode 122 and/or 124 can be formed as a continuous layer providing a ground plane or other potential and electrode 122 can be formed as a continuous layer in contact with a mechanical support layer (not shown), which can be formed from silicon or other suitable mechanical stiffening material. In still other embodiments, the electrode 126 can be routed along edge support 105. For example, when actuation voltage is applied to the electrodes, the membrane 110 will deform and move out of plane. The motion results in generation of an acoustic (ultrasonic) wave.

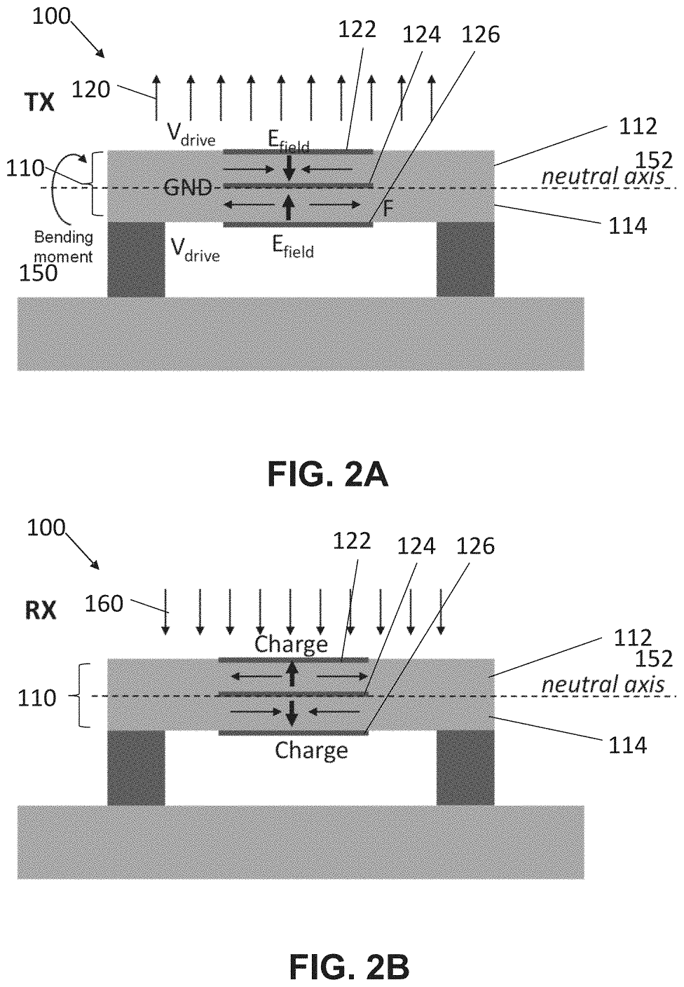

In some embodiments, electrodes 122 and 126 are coupled to a same terminal and operate as a single electrode, where electrode 124 is coupled to ground (GND) or other potential. FIGS. 2A and 2B illustrate the operation of dual layer ultrasonic transducer device in a transmit mode and a receive mode, respectively. FIG. 2A is a diagram illustrating an example transmit operation (e.g., transmit mode) of a dual layer ultrasonic transducer device 100, where the two piezoelectric layers 112 and 114 are activated during the transmit operation, according to some embodiments. In some embodiments, during the transmit operation, piezoelectric layer 112 and piezoelectric layer 114 are driven using the same drive voltage (V_{drive}) causing opposing electric fields (E_{field}) toward electrode 124 from electrode 122 and electrode 126 to generate an ultrasonic signal (illustrated as arrows 120). Applying the drive voltage to electrode 122 and electrode 126 also causes bending moment 150 about neutral axis 152. For instance, piezoelectric layers 112 and 114 are driven with electric fields in opposing directions, causing double the force (F) and double the deformation of a single piezo electric layer, resulting in doubling the pressure with respect to a single piezoelectric layer driven using the same drive voltage.

FIG. 2B is a diagram illustrating an example receive operation (e.g., receive mode) of a dual layer ultrasonic transducer device 100, where the two piezoelectric layers 112 and 114 are activated during the receive operation, according to some embodiments. In some embodiments, during the receive operation, the deformation of membrane 110 is induced by the incoming pressure (illustrated as arrows 160), causing charge to be collected at electrode 122 and electrode 126. For instance, electrode 122 and electrode 126 both receive charge, resulting in doubling the charge collected with respect to a single piezoelectric layer responsive to the same incoming pressure. Moreover, doubling the receive capacitance relative to a single piezoelectric layer makes dual layer ultrasonic transducer device 100 more robust to parasitic capacitance loss than a single layer ultrasonic transducer device. With reference to FIGS. 2A and 2B, electrodes 122 and 126 can be connected to a same terminal (or a separate terminal), where the underlying circuitry controls switching operation between the transmit operation and the receive operation.

With reference to FIG. 1, in some embodiments, electrodes 122 and 126 are coupled to different terminals and operate as separate electrodes, where electrode 124 is coupled to ground (GND) or other potential. For instance, piezoelectric layer 112 and electrodes 122 and 124 can be used during one of a receive or transmit operation, and piezoelectric layer 114 and electrodes 124 and 126 can be used during the other operation (e.g., piezoelectric layer 112 used for the transmit operation and piezoelectric layer 114 used for the receive operation, or vice versa).

Embodiments described herein also allow for the use of dual layer ultrasonic transducer device 100 in a differential drive or differential sense/receive mode. Description of embodiments in relation to FIG. 2A and FIG. 2B illustrates examples where electrode 124 is at a fixed potential, e.g., ground potential. In the dual drive mode, the electrodes on either side of a piezoelectric layer are used as drive electrodes that have inverse waveforms, meaning they both have a varying potential in order to generate the ultrasonic waves.

With the differential drive as discussed herein, with reference to FIG. 2A, electrodes 122 and 126 are driven with a potential having a waveform, and electrode 124 is driven with a potential having a waveform inverse to the waveform driving electrodes 122 and 126 (e.g., a phase shift, a 180 degree difference between a transmit state and a receive state). Other suitable phase shifts can also be applied depending, for example, on the transducer design. With the inverse polarity, the piezoelectric layers can be driven with more force as compared to electrode 124 being coupled to ground. The differential drive increases the electric field and therefore increase the transmit power of the transducer. The half duty cycle in both cases is V_{drive} and, therefore, twice the power can be output without inputting twice the amount of power. The voltage variation amplitude and used maximum and minimum voltage for the bottom and top electrodes can be identical, or they can be different. Accordingly, more signal can be achieved resulting in better sensor performance. The differential drive mode is used during the transmission phase, and the subsequent receive phase can use any of the variations discussed above. In addition to the differential drive mode, a differential receive mode can also be used (e.g., at FIG. 2B). For example, as the membrane flexes during receive, strain induced charges are generated across the piezoelectric layers. Due to the different polarity of the charges induced as a function of the direction of the bending strains, the electrodes can be designed according to the shape and location of these strains to capture the differential signals.

Further, provided is a dual layer ultrasonic transducer device 100 with differential drive and sense. For the differential drive, two or more electrodes used for the transmit operation can be driven with differential signal, providing more transmit pressure compared to a single-ended drive. For the differential sense, the electrodes used for the receive operation can be arranged such that the electrodes contact parts of the piezoelectric layer with out-of-phase stress. Taking as differential signal across these electrodes can help increase the receive signal. The different electrodes may be connected to different inputs of a differential amplifier in the sensing circuit.

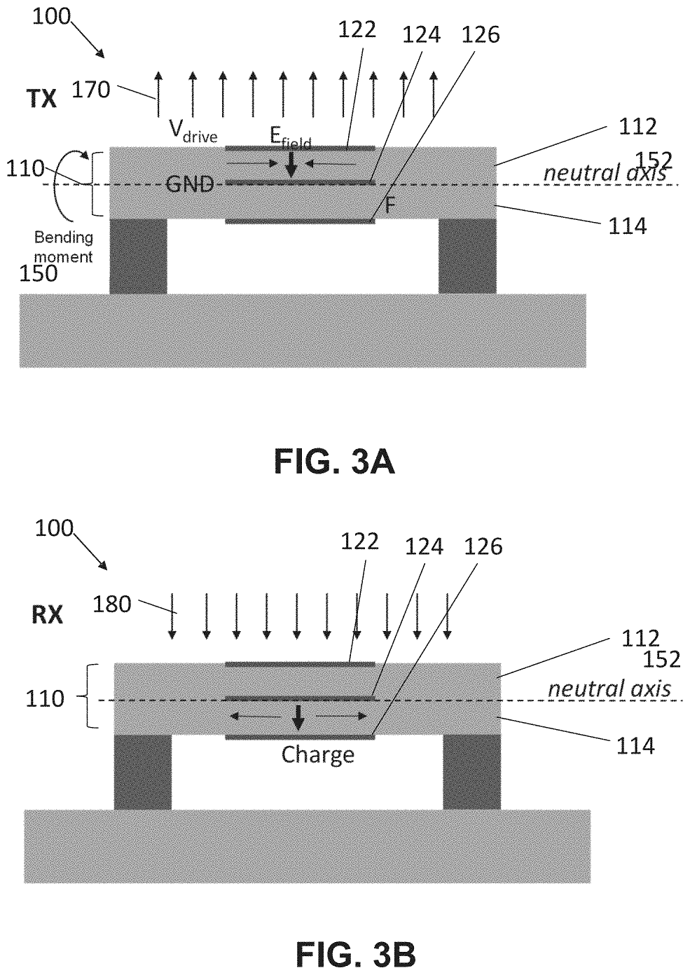

FIG. 3A is a diagram illustrating an example transmit operation of a dual layer ultrasonic transducer device 100, where one piezoelectric layer is activated during the transmit operation, according to some embodiments. In some embodiments, during the transmit operation, piezoelectric layer 112 is driven using drive voltage (V_{drive}) causing an electric field (E_{field}) toward electrode 124 from electrode 122 to generate an ultrasonic signal (illustrated as arrows 170). Applying the drive voltage to electrode 122 also causes bending moment 150 about neutral axis 152. It should be appreciated that electrode 126 and piezoelectric layer 114 can be used in a similar manner during the transmit operation. It should be appreciated that the described embodiments allows for one pair of electrodes to be used for the transmit operations and another pair of electrodes is used for the receive operation. The unused electrode during the transmit or receive operation can be coupled to a particular potential for shielding or can be floating. In other embodiments, electrodes 122 and 124 can be used to provide a differential drive mode.

FIG. 3B is a diagram illustrating an example receive operation of a dual layer ultrasonic transducer device 100, where piezoelectric layer 114 is activated during the receive operation, according to some embodiments. In some embodiments, during the receive operation, the deformation of membrane 110 is induced by the incoming pressure (illustrated as arrows 180), causing charge to be collected at electrode 126. It should be appreciated that electrode 122 and piezoelectric layer 112 can be used in a similar manner during the receive operation. In other embodiments, electrodes 124 and 126 can be used to provide a differential sense mode.

It should be appreciated that using different electrodes for the transmit operation and receive operation allows for a high voltage to be using during the transmit operation without needing to provide protection to receive componentry (e.g., a receive amplifier). Moreover, using piezoelectric layers for the transmit operation and receive operation allows for the use of different thicknesses and/or materials for each of the piezoelectric layers, thereby allowing for additional optimizations of dual layer ultrasonic transducer device 100 (e.g., each piezoelectric layer is optimized for its function).

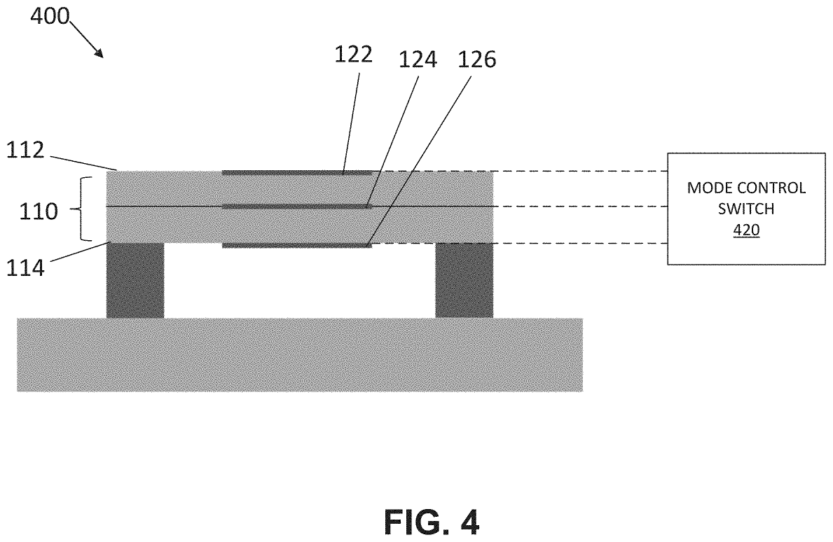

FIG. 4 is a diagram illustrating a dual layer ultrasonic transducer device 400 including a mode control switch 420 for switching between different modes of operating the transducer, e.g., activating one piezoelectric layer and activating two piezoelectric layers during a transmit and/or receive operation, according to various embodiments. Dual layer ultrasonic transducer device 400 operates in a similar manner, and includes the same configuration, as dual layer ultrasonic transducer device 100 of FIG. 1.

Dual layer ultrasonic transducer device 400 further includes mode control switch 420 (e.g., a switchable control) coupled to electrodes 122, 124, and 126, for example, for switching between a two terminal mode operating the first electrode and the second electrode as separate electrodes (e.g., as described in accordance with FIGS. 3A and 3B), and a one terminal mode operating the first electrode and the second electrode as a single electrode (e.g., as described in accordance with FIGS. 2A and 2B). In one embodiment, mode control switch 420 is a high voltage switch. It should be appreciated that mode control switch 420 can be electrically coupled to electrodes 122, 124, and 126 through any type of electrical connect that runs through or along the substrate, edge support, or membrane, and that the illustrated example is merely a schematic indicating the connection between mode control switch 420 and electrodes 122, 124, and 126. Different modes may provide different advantages and disadvantages, and mode control switch 420 may control the different modes based on the context or requirements. For example, by switching between different modes, the power, sensitivity, and/or gain can be controlled, and used to adapt to higher or lower signal strength (e.g., as a function of depth of the signal). In a device with a plurality of transducers, different groups of transducers, or sections of the device, may operate in different modes depending on requirements. For example, for contact surfaces with a varying thickness, different regions with different thicknesses may used different modes.

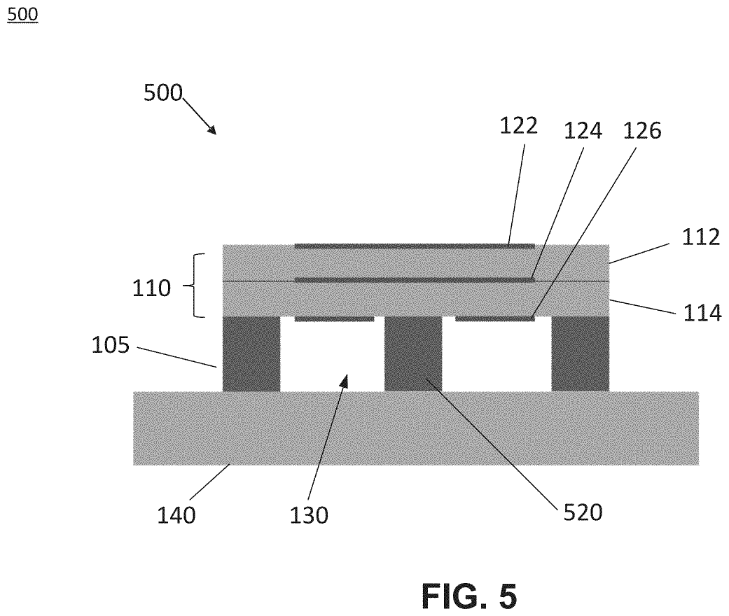

FIG. 5 is a diagram illustrating a dual layer ultrasonic transducer device 500 having an interior support 520 coupled to the substrate 140 and the membrane 110, according to some embodiments. Dual layer ultrasonic transducer device 500 operates in a similar manner, and includes substantially the same configuration, as dual layer ultrasonic transducer device 100 of FIG. 1, apart from the addition of interior support 520.

Dual layer ultrasonic transducer device 500 includes an interior pinned membrane 110 positioned over a substrate 140 to define a cavity 130. In one embodiment, membrane 110 is attached both to a surrounding edge support 105 and interior support 520. Interior supports 520 may also be referred to as “pinning structures,” as they operate to pin the membrane 110 to the substrate 140. It should be appreciated that interior support 520 may be positioned anywhere within cavity 130 of a dual layer ultrasonic transducer device 500, may have any type of shape (or variety of shapes), and that there may be more than one interior support 520 within dual layer ultrasonic transducer device 500.

Interior support 520 may be made of electrically conducting materials, such as and without limitation, aluminum, molybdenum, or titanium. Interior support 520 may also be made of dielectric materials, such as silicon dioxide, silicon nitride or aluminum oxide that have electrical connections along the sides of or in vias through edge support 105 or interior support 520, electrically coupling electrode 126 to electrical wiring in substrate 140.

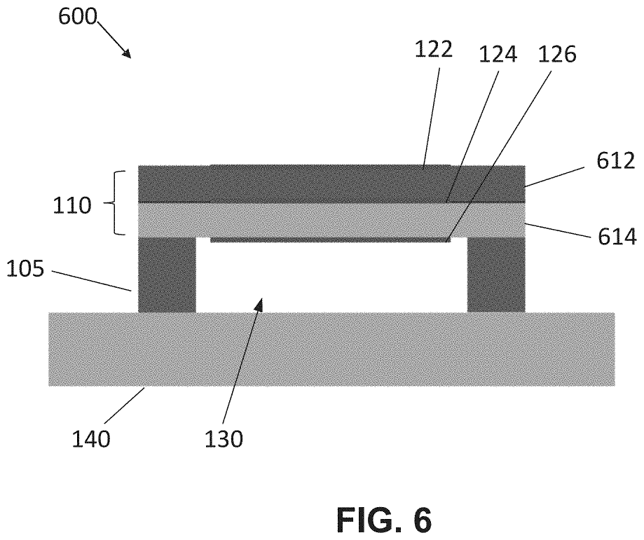

FIG. 6 is a diagram illustrating a dual layer ultrasonic transducer device 600 having piezoelectric layers comprised of different materials, according to some embodiments. Dual layer ultrasonic transducer device 600 operates in a similar manner, and includes the same configuration, as dual layer ultrasonic transducer device 100 of FIG. 1, apart from the explicit use of different materials in piezoelectric layers 612 and 614.

In various embodiments, the dual layer ultrasonic transducer device 600 employs piezoelectric layers 612 and 614, comprised of materials such as, but not limited to, aluminum nitride (AlN), scandium doped aluminum nitride (ScAlN), lead zirconate titanate (PZT), quartz, polyvinylidene fluoride (PVDF), and/or zinc oxide, to facilitate both acoustic signal production and sensing, where piezoelectric layers 612 and 614 are comprised of different materials. For instance, utilizing different materials in piezoelectric layers 612 and 614 allows for the optimization of each piezoelectric layer according to its usage.

For example, where piezoelectric layer 612 is utilized for a transmit operation, a piezoelectric material that provides beneficial performance during transmission of ultrasonic signals, such as PZT, can be used in piezoelectric layer 612. Similarly, where piezoelectric layer 614 is utilized for a receive operation, a piezoelectric material that provides beneficial performance during receipt of ultrasonic signals, such as AlN, can be used in piezoelectric layer 614.

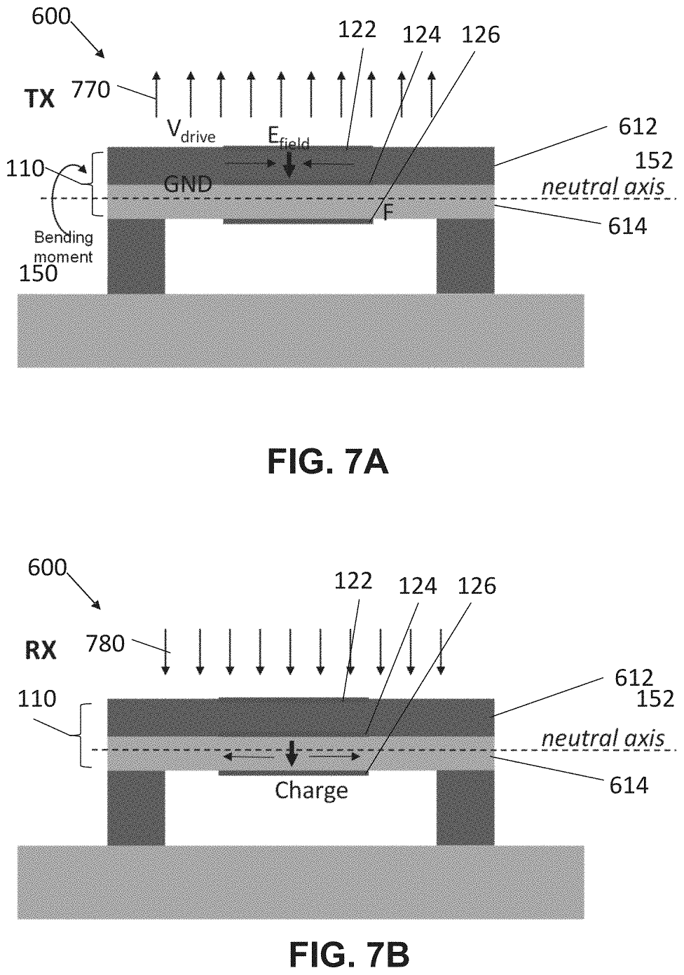

FIG. 7A is a diagram illustrating an example transmit operation of a dual layer ultrasonic transducer device 600 having piezoelectric layers comprised of different materials, according to some embodiments. In some embodiments, during the transmit operation, piezoelectric layer 612 is driven using drive voltage (V_{drive}) causing an electric field (E_{field}) toward electrode 124 from electrode 122 to generate an ultrasonic signal (illustrated as arrows 770). Applying the drive voltage to electrode 122 also causes bending moment 150 about neutral axis 152. It should be appreciated that electrode 126 and piezoelectric layer 614 can be used in a similar manner during the transmit operation. In other embodiments, electrodes 122 and 124 can be used to provide a differential drive mode. It should be appreciated that using piezoelectric layers 612 and 614 comprised of different materials allows for controlling the location of neutral axis 152. Neutral axis 152 is positioned non-symmetrically within membrane 110 (e.g., positioned within piezoelectric layer 614), rather than in the middle of membrane 110.

FIG. 7B is a diagram illustrating an example receive operation of a dual layer ultrasonic transducer device 600 having piezoelectric layers comprised of different materials, according to some embodiments. In some embodiments, during the receive operation, the deformation of membrane 110 is induced by the incoming pressure (illustrated as arrows 780), causing charge to be collected at electrode 126. It should be appreciated that electrode 122 and piezoelectric layer 612 can be used in a similar manner during the receive operation. It should be appreciated that using different materials in piezoelectric layers 612 and 614 allows for additional optimization of dual layer ultrasonic transducer device 600. In other embodiments, electrodes 124 and 126 can be used to provide a differential sense mode.

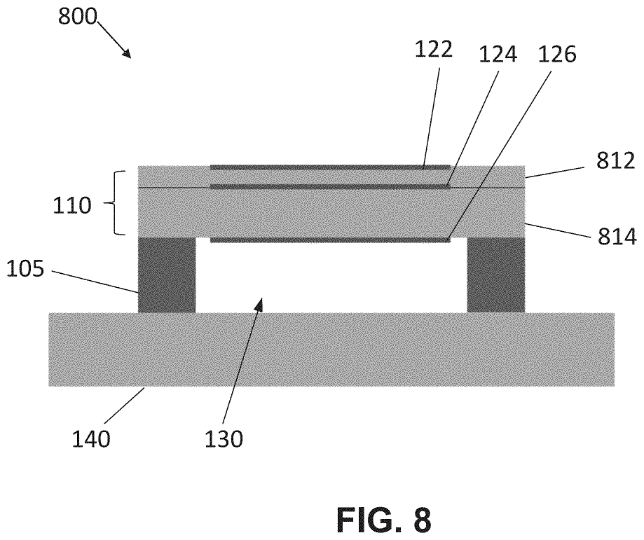

FIG. 8 is a diagram illustrating a dual layer ultrasonic transducer device 800 having piezoelectric layers having different thicknesses, according to some embodiments. Dual layer ultrasonic transducer device 800 operates in a similar manner, and includes the same configuration, as dual layer ultrasonic transducer device 100 of FIG. 1, apart from the explicit use of different thicknesses in piezoelectric layers 812 and 814.

In various embodiments, the dual layer ultrasonic transducer device 800 employs piezoelectric layers 812 and 814, where piezoelectric layers 812 and 814 have different thicknesses. For instance, utilizing different thicknesses for piezoelectric layers 812 and 814 allows for the optimization of each piezoelectric layer according to its usage. For example, where piezoelectric layer 812 is utilized for a transmit operation, piezoelectric layer 812 can be a thinner layer than piezoelectric layer 814, where a thinner layer is better for improved performance of the transmit operation. Similarly, where piezoelectric layer 814 is utilized for a receive operation, piezoelectric layer 814 can be a thicker layer than piezoelectric layer 812, where a thicker layer is better for improved performance of the receive operation.

It should be appreciated that using piezoelectric layers of different thicknesses and or different materials, as illustrated in FIGS. 6 and 8, allows for controlling the location of the neutral axis (e.g., neutral axis 152 of FIGS. 7A and 7B). The neutral axis may be positioned non-symmetrically (e.g., positioned other than in the middle of the membrane) within the membrane to improve transmission efficiency or receiving sensitivity, depending on the application or context of use of the ultrasonic transducer. As such, the neutral axis may be located at different location in either of the piezoelectric layers, or in the buffer layer, if present. For example, the neutral axis can be moved towards one of the outer electrodes to improve performance of the ultrasonic transducer.

FIG. 9 is a diagram illustrating a dual layer ultrasonic transducer device having a buffer layer between the two piezoelectric layers, according to some embodiments. Dual layer ultrasonic transducer device 900 operates in a similar manner, and includes the same configuration, as dual layer ultrasonic transducer device 100 of FIG. 1, apart from the addition of buffer layer 920 and electrode 915 in membrane 910. The different embodiments and modes discussed in relation to transducers without a central buffer layer may also be applied here for at least one of the piezoelectric layers 112 and 114. In accordance with various embodiments, piezoelectric layers 112 and 114 and buffer layer 920 have thicknesses in the range of one to ten microns. For example, piezoelectric layers 112 and 114 have a thickness of one micron and buffer layer 920 have thickness of two microns, such that membrane 110 has a thickness of four microns).

Dual layer ultrasonic transducer device 900 includes a membrane 910 positioned over a substrate 140 to define a cavity 130. In one embodiment, membrane 910 is attached to a surrounding edge support 105. Membrane 910 comprises multiple piezoelectric layers and buffer layer 920. In an example embodiment, the membrane 910 includes piezoelectric layers 112 and 114, buffer layer 920, and electrodes 122, 126, 912, and 914, with electrodes 122 and 912 on opposing sides of piezoelectric layer 112, electrodes 914 and 126 on opposing sides of piezoelectric layer 114, and buffer layer 920 between electrodes 912 and 914.

Buffer layer 920 separates piezoelectric layers 112 and 114. Buffer layer 920 can be comprised of materials such as, but not limited to, silicon, silicon oxide, polysilicon, silicon nitride, or any non-conducting oxide layer (or stacks of layers). Moreover, it should be appreciated that the buffer material can be application specific, e.g., selected based on a desired frequency of operation of dual layer ultrasonic transducer device 900. For example, buffer layer 920 can be a metal. It should be appreciated that the stiffer the material of buffer layer 920, the higher the frequency.

Buffer layer 920 allows for improved tuning of the transmit and receive operations, by enhancing the performance of the transmit and receive operations. The frequency can be tuned according to thickness of buffer layer 920 so as to optimize the thicknesses of piezoelectric layers 112 and 114 to improve the figure of merit (FOM) of dual layer ultrasonic transducer device 900. Moreover, the neutral axis can be designed to not be in the middle of membrane 910 so as to achieve a better FOM. Buffer layer 920 also supports tuning of the thicknesses and materials of piezoelectric layers 112 and 114.

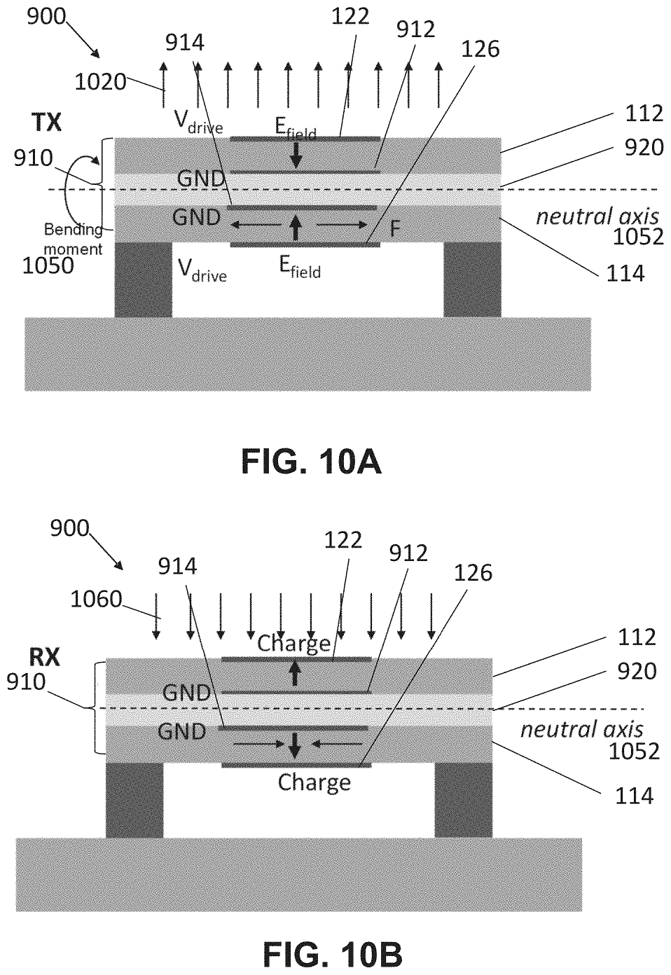

In some embodiments, electrodes 122 and 126 are coupled to a same terminal and operate as a single electrode, where electrodes 912 and 914 are coupled to ground (GND). FIG. 10A is a diagram illustrating an example transmit operation of a dual layer ultrasonic transducer device 900 having buffer layer 920 between piezoelectric layers 112 and 114, according to some embodiments. In some embodiments, during the transmit operation, piezoelectric layer 112 and piezoelectric layer 114 are driven using the same drive voltage (V_{drive}) causing opposing electric fields (E_{field}) toward electrode 912 from electrode 122 and toward electrode 914 from electrode 126 to generate an ultrasonic signal (illustrated as arrows 1020). Applying the drive voltage to electrode 122 and electrode 126 also causes bending moment 1050 about neutral axis 1052. The use of buffer layer 920 provides for an increase the bending moment 1050 by increasing the mechanical leverage from a piezoelectric center to the neutral axis 1052. For instance, piezoelectric layers 112 and 114 are driven with electric fields in opposing directions, causing double the force (F) and double the deformation of a single piezo electric layer, resulting in doubling the pressure with respect to a single piezoelectric layer driven using the same drive voltage. In other embodiments, at least one of electrode pair 122 and 912 and electrode pair 126 and 914 can be used to provide a differential drive mode.

FIG. 10B is a diagram illustrating an example receive operation of a dual layer ultrasonic transducer device 900 having buffer layer 920 between piezoelectric layers 112 and 114, according to some embodiments. In some embodiments, during the receive operation, the deformation of membrane 910 is induced by the incoming pressure (illustrated as arrows 1060), causing charge to be collected at electrode 122 and electrode 126. For instance, electrode 122 and electrode 126 both receive charge, resulting in doubling the charge collected with respect to a single piezoelectric layer responsive to the same incoming pressure. Moreover, doubling the receive capacitance relative to a single piezoelectric layer makes dual layer ultrasonic transducer device 900 more robust to parasitic capacitance loss than a single layer ultrasonic transducer device. In other embodiments, at least one of electrode pair 122 and 912 and electrode pair 126 and 914 can be used to provide a differential sense mode.

With reference to FIG. 9, in some embodiments, electrodes 122 and 126 are coupled to different terminals and operate as separate electrodes, where electrodes 912 and 914 are coupled to ground (GND). For instance, piezoelectric layer 112 and electrodes 122 and 912 can be used during one of a receive or transmit operation, and piezoelectric layer 114 and electrodes 914 and 126 can be used during the other operation (e.g., piezoelectric layer 112 used for the transmit operation and piezoelectric layer 114 used for the receive operation, or vice versa).

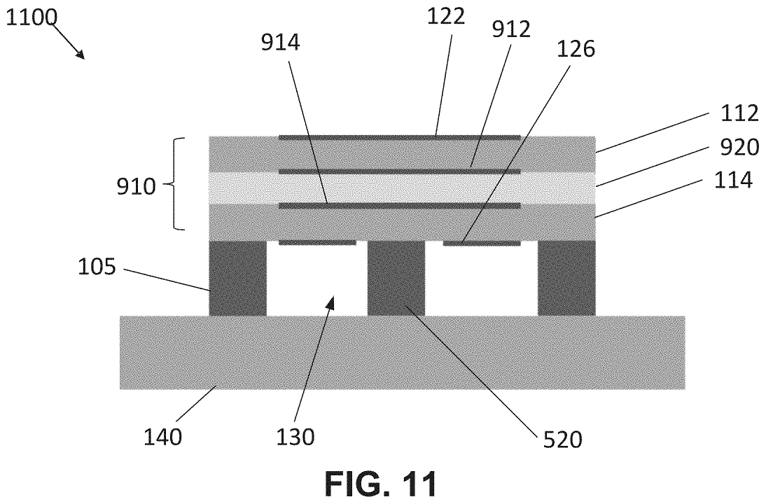

FIG. 11 is a diagram illustrating a dual layer ultrasonic transducer device 1100 having a buffer layer 920 between piezoelectric layers 112 and 114, and an interior support 520 coupled to substrate 140 and membrane 910, according to some embodiments. Dual layer ultrasonic transducer device 1100 operates in a similar manner, and includes the same configuration, as dual layer ultrasonic transducer device 100 of FIG. 1, apart from the addition of buffer layer 920 and electrodes 912 and 914 in membrane 910, and interior support 520.

What has been described above includes examples of the subject disclosure. It is, of course, not possible to describe every conceivable combination of components or methodologies for purposes of describing the subject matter, but it is to be appreciated that many further combinations and permutations of the subject disclosure are possible. Accordingly, the claimed subject matter is intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated examples of the claimed subject matter.

The aforementioned systems and components have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components. Any components described herein may also interact with one or more other components not specifically described herein.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

Thus, the embodiments and examples set forth herein were presented in order to best explain various selected embodiments of the present invention and its particular application and to thereby enable those skilled in the art to make and use embodiments of the invention. However, those skilled in the art will recognize that the foregoing description and examples have been presented for the purposes of illustration and example only. The description as set forth is not intended to be exhaustive or to limit the embodiments of the invention to the precise form disclosed.

What is claimed is:

1. An ultrasonic transducer device comprising:
a substrate;
an edge support structure connected to the substrate;
a membrane connected to the edge support structure such that a cavity is defined between the membrane and the substrate, the membrane configured to allow movement at ultrasonic frequencies, the membrane comprising:
a first piezoelectric layer having a first surface and a second surface;
a second piezoelectric layer having a first surface and a second surface,
wherein the second surface of the first piezoelectric layer faces the first surface of the second piezoelectric layer;
a first electrode coupled to the first surface of the first piezoelectric layer;
a second electrode coupled to the second surface of the second piezoelectric layer; and
a third electrode between the first piezoelectric layer and the second piezoelectric layer; and
an interior support structure disposed within the cavity and connected to the substrate and the membrane.

2. The ultrasonic transducer device of claim 1, wherein the first electrode and the second electrode are coupled to a same terminal and operate as a single electrode.

3. The ultrasonic transducer device of claim 2, wherein during a transmit mode the first piezoelectric layer and the second piezoelectric layer are driven by opposing electric fields from the first electrode and the second electrode to generate an ultrasonic signal, and during a receive mode charge is collected by the first electrode and the second electrode.

4. The ultrasonic transducer device of claim 1, wherein the first electrode and the second electrode are coupled to separate terminals and operate as separate electrodes.

5. The ultrasonic transducer device of claim 4, wherein the first electrode and the third electrode operate collectively in a transmit mode and the second electrode and the third electrode operate collectively in a receive mode.

6. The ultrasonic transducer device of claim 4, wherein the first electrode and the third electrode operate collectively in a receive mode and the second electrode and the third electrode operate collectively in a transmit mode.

7. The ultrasonic transducer device of claim 1, further comprising:
a switchable control coupled to the first electrode and the second electrode for switching between a two terminal mode operating the first electrode and the second electrode as separate electrodes and a one terminal mode operating the first electrode and the second electrode as a single electrode.

8. The ultrasonic transducer device of claim 1, wherein the first piezoelectric layer and the second piezoelectric layer have different thicknesses.

9. The ultrasonic transducer device of claim 1, wherein the first piezoelectric layer and the second piezoelectric layer are comprised of different materials.

10. The ultrasonic transducer device of claim 1, further comprising:
a buffer layer between the first piezoelectric layer and the second piezoelectric layer; and
a fourth electrode between the second piezoelectric layer and the buffer layer;
wherein the third electrode is between the first piezoelectric layer and the buffer layer.

11. The ultrasonic transducer device of claim 1, wherein a neutral axis of the ultrasonic transducer device is positioned non-symmetrically within the membrane.

12. An ultrasonic transducer device comprising:
a substrate;
an edge support structure connected to the substrate;
a membrane connected to the edge support structure such that a cavity is defined between the membrane and the substrate, the membrane configured to allow movement at ultrasonic frequencies, the membrane comprising:
a first piezoelectric layer having a first surface and a second surface;
a second piezoelectric layer having a first surface and a second surface, wherein the second surface of the first piezoelectric layer faces the first surface of the second piezoelectric layer;
a buffer layer between the first piezoelectric layer and the second piezoelectric layer;
a first electrode coupled to the first surface of the first piezoelectric layer;
a second electrode coupled to the second surface of the second piezoelectric layer;
a third electrode between the first piezoelectric layer and the buffer layer; and
a fourth electrode between the second piezoelectric layer and the buffer layer.

13. The ultrasonic transducer device of claim 12, wherein the first electrode and the second electrode are coupled to a same terminal and operate as a single electrode.

14. The ultrasonic transducer device of claim 13, wherein during a transmit mode the first piezoelectric layer and the second piezoelectric layer are driven by opposing electric fields from the first electrode and the second electrode to generate an ultrasonic signal, and during a receive mode charge is collected by the first electrode and the second electrode.

15. The ultrasonic transducer device of claim 12, wherein the first electrode and the second electrode are coupled to separate terminals and operate as separate electrodes.

16. The ultrasonic transducer device of claim 15, wherein the first electrode and the third electrode operate collectively in a transmit mode and the second electrode and the fourth electrode operate collectively in a receive mode.

17. The ultrasonic transducer device of claim 15, wherein the first electrode and the third electrode operate collectively in a receive mode and the second electrode and the fourth electrode operate collectively in a transmit mode.

18. The ultrasonic transducer device of claim 12, further comprising:
a switchable control coupled to the first electrode and the second electrode for switching between a two terminal mode operating the first electrode and the second electrode as separate electrodes and a one terminal mode operating the first electrode and the second electrode as a single electrode.

19. The ultrasonic transducer device of claim 12, wherein the first piezoelectric layer and the second piezoelectric layer have different thicknesses.

20. The ultrasonic transducer device of claim 12, wherein the first piezoelectric layer and the second piezoelectric layer are comprised of different materials.

21. The ultrasonic transducer device of claim 12, further comprising:
an interior support structure disposed within the cavity and connected to the substrate and the membrane.

22. The ultrasonic transducer device of claim 12, wherein a neutral axis of the ultrasonic transducer device is positioned non-symmetrically within the membrane.
